(19) United States
(12) Patent Application Publication
(10) Pub. No.: US 2008/0092887 A1
(43) Pub. Date: Apr. 24, 2008

(54) Single-Dose Inhalation Devices

(76) Inventors: Peter D. Hodson, Derbyshire (GB); Graham R. Purkins, Loughborough (GB); Stephen J. Howgill, Thurcaston (GB)

Correspondence Address: 3M INNOVATIVE PROPERTIES COMPANY, PO BOX 33427, ST. PAUL, MN, 55133-3427, US

(21) Appl. No.: 11/720,503

(22) PCT Filed: Nov. 9, 2005
(86) PCT No.: PCT/US05/40393

§ 371 Date: May 30, 2007

(30) Foreign Application Priority Data
Dec. 7, 2004 (GB) 0426780.3

(51) Int. Cl. A61M 15/00

(52) U.S. Cl. 128/203.21

(57) ABSTRACT

An inhalation device comprising a hermetically sealed receptacle (1) containing a single dose of a pressurized formulation (30) comprising a pharmaceutically active ingredient and a liquefied aerosol propellant consisting of HFA 134a, HFA 227 or a mixture thereof and wherein at least a portion (12) of the receptacle is perforable.

This invention relates generally to single-dose inhalation devices comprising a single hermetically sealed receptacle holding a single pre-metered dose of pressurized liquefied propellant-based formulation.

Pressurized metered dose inhalers have been used for over forty years for the treatment of asthma and other respiratory conditions. Pressurized metered dose inhalers comprise a container filled with many doses of propellant-based formulation, together with a metering valve for dispensing individual metered doses upon demand. One of the disadvantages of conventional metered dose inhalers is the difficulty in providing a low number of doses (e.g. less than thirty) that is appropriate for some therapy regimes. Furthermore, although dry powder inhalation devices or liquid nasal devices, which sometimes provide individual doses of formulation, are commercially available and although a single dose inhalation device including gases such as carbon dioxide, oxygen or nitrogen has been proposed (see U.S. Pat. No. 4,137,914 published in 1979), to date no commercially viable single dose pressurized inhalation device has been proposed or commercialized.

### SUMMARY OF THE INVENTION

There is a need to provide a commercially viable pressurized single dose inhalation device suitable for delivering a pharmaceutically active ingredient to the lung.

We have found that through the use of a single hermetically sealed receptacle containing a single dose of a pressurized formulation comprising a pharmaceutically active ingredient and a liquefied aerosol propellant consisting of 1,1,1,2-tetrafluoroethane (HFA 134a), 1,1,1,2,3,3,3-heptafluoropropane (HFA 227) or a mixture thereof and wherein at least a portion of the receptacle is perforable (i.e. capable of being perforated), an inhalation device is provided in which upon actuation of the device and thus perforation of the receptacle, an aerosol is generated allowing for delivery of the pharmaceutically active ingredient to the lung. Moreover, the generated aerosol for inhalation advantageously comprises fine droplets of liquefied propellant together with pharmaceutically active ingredient allowing for reliable delivery and transport of the active ingredient to the patient's lungs.

Thus the present invention provides an inhalation device comprising a hermetically sealed receptacle containing a single dose of a pressurized formulation comprising a pharmaceutically active ingredient and a liquefied aerosol propellant consisting of HFA 134a, HFA 227 or a mixture thereof and wherein at least a portion of the receptacle is perforable.

In the previously proposed pressurized single dose devices (see U.S. Pat. No. 4,137,914 and U.S. Pat. No. 6,602,213) including e.g. compressed gases such as carbon dioxide, oxygen or nitrogen, upon actuation (or activation) of said system, the gas immediately vaporizes thus generating a “hard” aerosol with just active ingredient with the devices being unsuitable for delivery of medicament to the lung or the correct portion thereof. In the devices proposed in U.S. Pat. No. 4,137,914 it has been recognized that much of the medicament will simply be deposited on to the internal surface of the mouthpiece (if in fact the dose is able to pass through the disclosed capillary tube prior to occlusion thereof. In the devices disclosed in U.S. Pat. No. 6,602,213, the device is to be provided with sufficiently high vapor pressure at room temperature such that proactive delivery means vaporize thoroughly upon activation (providing a hard aerosol with just active ingredient) and similar to the devices disclosed in US'914 much of the medicament will simply be immediately deposited on surfaces of the device and/or retained within the device.

The receptacles of the devices described herein preferably have relatively low internal pressure within the receptacle at ambient temperature (22° C.), e.g. at most 7 atmospheres pressure absolute. This is advantageous inter alia in terms of cost effectiveness and ease in manufacture, in that the receptacle need not be spheroid in shape (a form typically necessary for containers having a high internal pressure). Also, favorably the at least perforable portion of the receptacle may be provided as a substantially planar portion to help avoid deflection or skidding of a firing pin used to perforate the receptacle. Such perforation is preferably rapid, in order to avoid or minimize unwanted aerosol leakage during perforation. Receptacles also desirably have an internal volume of less than 0.3 ml. The volume of the pressurized formulation is typically 150 μl at most.

Devices in accordance with the invention preferably further comprise a firing pin, wherein the firing pin comprises a channel having an opening at both ends, the first end positioned towards the receptacle, more desirably the channel is defined by an internal surface of the firing pin. In particular to provide a reliable and rapid firing force for perforating the receptacle (independent of any force provided by the patient), the receptacle and firing pin are desirably mutually biased towards one another, e.g. through the use of a compression spring. In this case the device is arranged such that in its stored position the receptacle and firing pin are retained apart and upon actuation of the device said retention is released such that receptacle and firing pin are mutually displaced to cause the firing pin to perforate the receptacle at said at least perforable portion and to cause aerosol formulation to pass through said channel and to the patient. The term “mutually biased” means that the receptacle is biased towards the firing pin, or the firing pin is biased towards the receptacle, or both the receptacle and firing pin are biased towards each other. The term “mutually displaced” means that the receptacle is displaced towards the firing pin, or the firing pin is displaced towards the receptacle, or both the receptacle and firing pin are displaced towards each other. To avoid deflection by or skidding of the firing pin during actuation, the receptacle preferably is biased towards the firing pin with the firing pin being held fixed within the device. In this case in the stored position of the device the receptacle is retained apart from the firing pin and upon actuation of the device said retention is released such that receptacle is displaced towards the firing pin to cause the firing pin to perforate the receptacle at said at least perforable portion and to cause aerosol formulation to pass through said channel and to the patient. It is preferable that the device is arranged such that upon perforation of said at least perforable portion of the receptacle by the firing pin, the first end of the channel of the firing pin passes into a liquid portion of the pressurized formulation.

To minimize occlusion within the channel of the firing pin the channel is preferably not provided in the form of a capillary tube. The internal surface of the said firing pin channel may be arranged to provide an expansion chamber, i.e. a chamber through which the expanding aerosol passes after its release from the perforated receptacle and before its subsequent passage through a more restrictive region such as a spray break-up orifice. To further minimize the potential for occlusion, the internal surface of said channel may advantageously be generally conical from the first end to the second end. Desirably the channel of the firing pin extends generally along a single axis. Typically the outermost surface of the firing pin positioned towards the receptacle (e.g. the outermost portion of the tip of the firing pin) is provided with a piercer capable of perforating the at least perforable portion of the receptacle. In order to avoid or minimize unwanted channel obstruction caused by any deformation of the firing pin and/or undesirable aerosol leakage upon perforation, it has been found advantageous to slightly offset the inlet of the channel from said outermost surface of the firing pin. Thus in preferred embodiments the first end of the channel of the firing pin is set back from said outermost surface and positioned adjacent to said piercer.

Devices in accordance with the invention suitably further comprise an outlet adapted for insertion into a patient's mouth or nose (nostril or nostrils) having a passageway. In order to provide compact devices, it is desirable that the channel of the firing pin defines substantially a single axis and the outlet passageway extends substantially along or substantially parallel to that axis.

Preferably devices are designed to be disposable after a single use, i.e. non-refillable for example with a second (replacement) receptacle. Devices described herein may be actuated either manually (e.g. by pressing a button or lever) or by inhaling (i.e. by breath-actuation). Breath-actuation is preferred. For example, the delivery of a pharmaceutically active ingredient for treatment of a systemic disease will tend to require the aerosol to penetrate the deep lung (e.g. to the alveolar regions). This requires good coordination of dose release with the early part of the patient's inspiratory maneuver. Breath-actuation provides a reliable way of ensuring such coordination, particularly for patients using an inhaler for the first time or for a one-time treatment or for patients using an inhaler very infrequently.

The dependent claims define further embodiments of the invention.

The invention, its embodiments and further advantages will be described in the following with reference to the following drawings or figures.

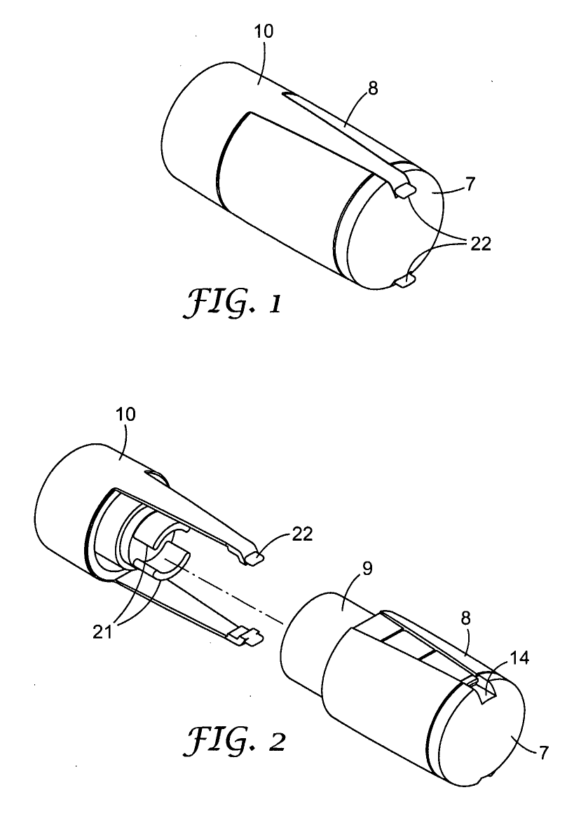

FIGS. 1 and 2 show perspective views of an exemplary embodiment of a device in accordance with the present invention.

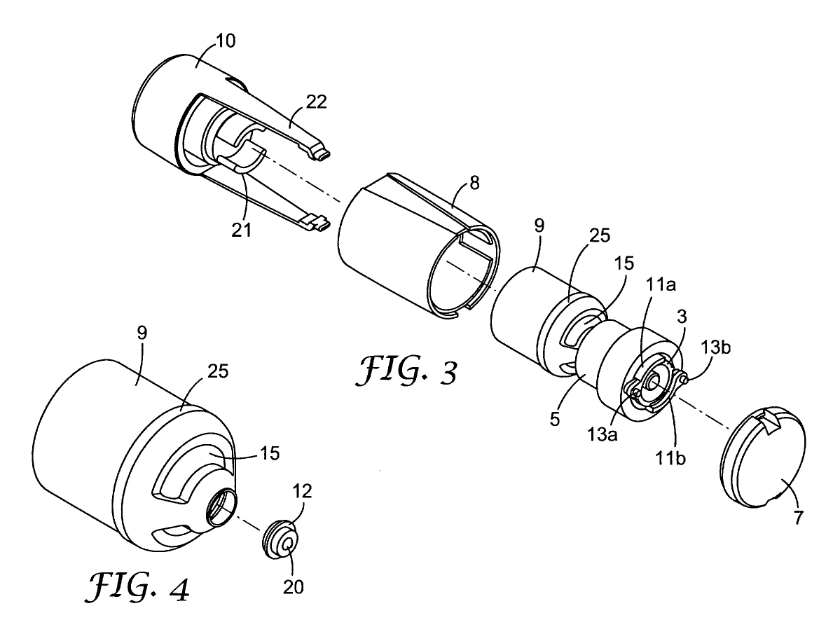

FIG. 3 represents an exploded diagram of the embodiment shown in FIGS. 1 and 2, while FIGS. 4 to 6 represent exploded diagrams of particular sub-assemblies of the embodiment.

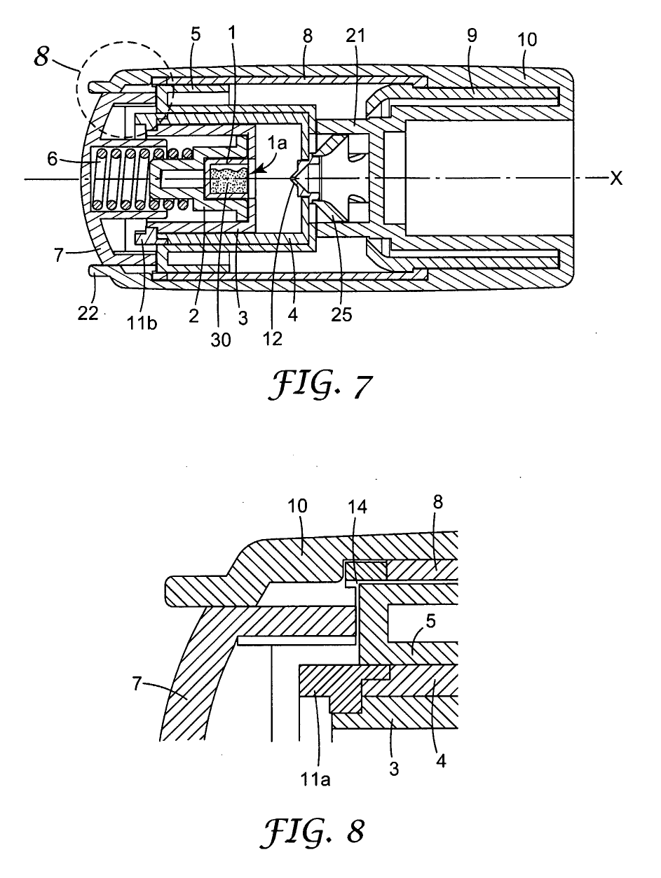

FIG. 7 represents a vertical cross-section of the embodiment as shown in FIG. 1, while FIG. 8 represents an enlargement of a portion of FIG. 7 (encircled in FIG. 7).

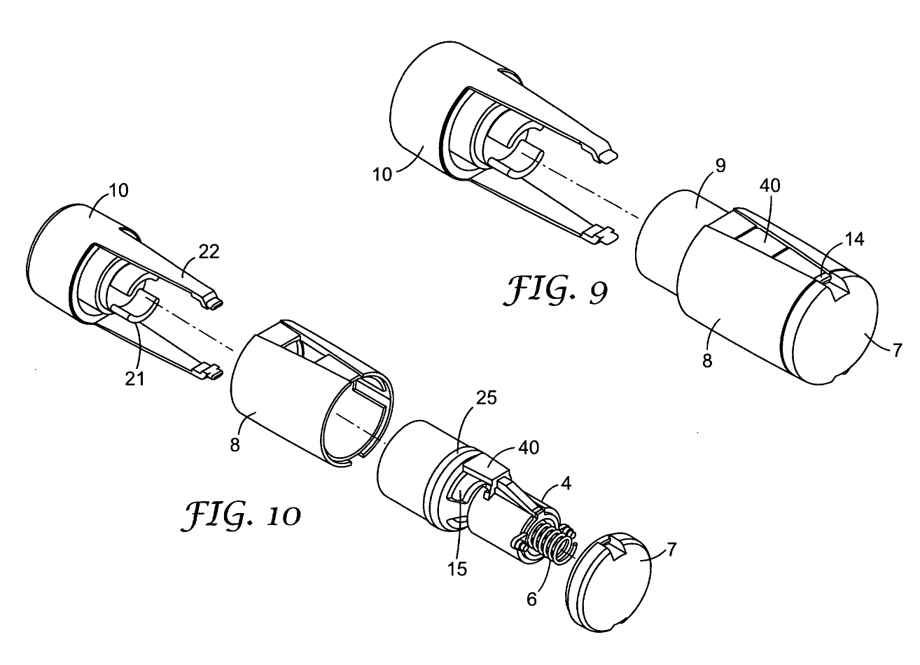

FIG. 9 shows a perspective view of another exemplary embodiment of a device in accordance with the present invention.

FIG. 10 represents an exploded diagram of the embodiment shown in FIG. 9, while FIG. 11 represents an exploded diagram of a particular sub-assembly of the embodiment.

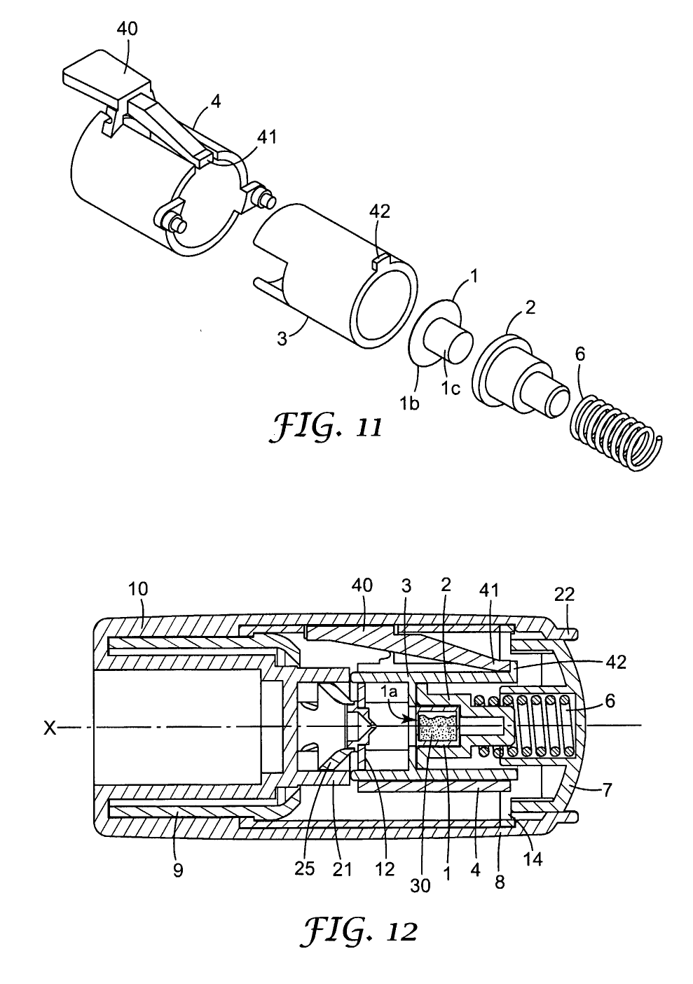

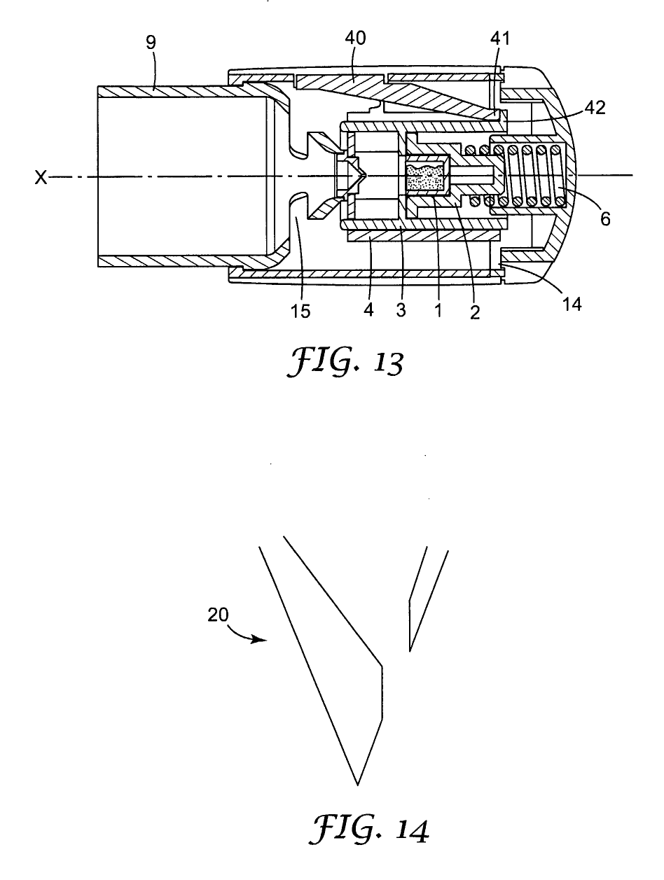

FIGS. 12 and 13 represent vertical cross-sections of the embodiment shown in FIG. 9 to 11 with and without a removable cover, respectively.

FIG. 14 represents an enlarged, schematic cross-section of a preferred formation of a portion of a tip of a firing pin for both the exemplary embodiments shown in FIGS. 1 to 8 and 9 to 13, respectively.

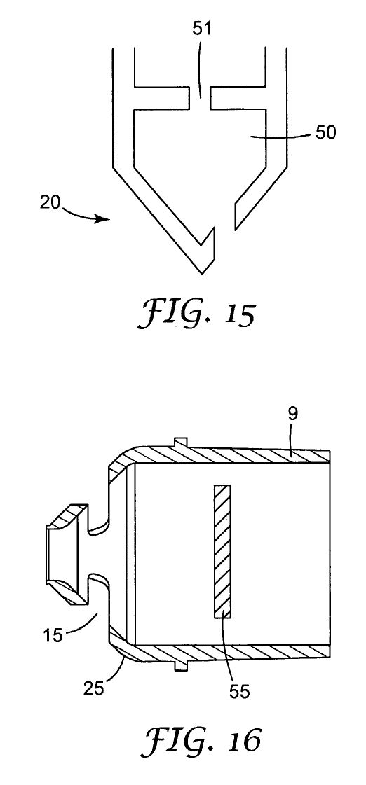

FIG. 15 represents an enlarged, schematic cross-section of an alternative preferred formation of a portion of a tip of a firing pin for both the exemplary embodiments shown in FIGS. 1 to 8 and 9 to 13, respectively.

FIG. 16 represents an enlarged, schematic cross-section of an alternative formation of an outlet region in the form of a mouthpiece suitable for use in both of the exemplary embodiments shown in FIGS. 1 to 8 and 9 to 13, respectively.

It is to be understood that not all the Figures are provided at the same scale.

It is also to be understood that the present invention covers all combinations of suitable, favorable, particular, desirable, advantageous, and preferred aspects of the invention described herein.

FIGS. 1 to 8 illustrate a first exemplary embodiment in accordance with the present invention. This exemplary embodiment is a disposable breath-actuated single-dose inhalation device. Referring to FIG. 7, this inhalation device comprises a hermetically sealed receptacle (1) comprising at least a perforable portion (1a), which as can be better viewed in FIG. 6. The perforable portion may advantageously be in the form of a foil (1b), in particular a metal foil, which is for example laser welded (as described in our co-pending application GB 0418738 filed Aug. 23, 2004, incorporated herein by reference) onto a receptacle body (1c), in particular a metal receptacle body. The receptacle contains a single dose of a pressurized formulation (30) comprising a pharmaceutically active ingredient and a liquefied aerosol propellant consisting of HFA 134a, HFA 227 or a mixture thereof.

As can be appreciated from FIG. 7, receptacles used in inhalation devices described herein are desirably free of elastomeric seals and diaphragms and/or dispensing valves, which is advantageous in avoiding leaking during storage as well as any ingress of air or moisture from the outside environment and/or any undesirable interaction with seal and/or diaphragm materials. Also as can be appreciated from FIG. 7, receptacles are desirably appropriately dimensioned (e.g. having a low or a minimum amount of head space) for containing a single dose of HFA-134a and/or HFA-227 based medicinal formulation. To accommodate a single dose of such a pharmaceutically active aerosol formulation, the receptacle desirably has an internal volume of less than 0.3 ml, more desirably 0.2 ml or less, even more desirably 0.15 ml or less, most desirably about 0.15 ml. Suitably the internal volume of the receptacle is at least 0.1 ml. As mentioned above the volume of pressurized formulation is typically 150 μl at most, and more desirably about 100 μl at most, and most desirably about 80 μl at most. Typically the pressurized formulation has a volume of at least about 25 μl, and more desirably at least about 40 μl and most desirably at least about 50 μl. Also mentioned above, the internal pressure within the receptacle is desirably at most 7 atmospheres absolute at ambient temperature, more desirably at most about 6.5 atmospheres absolute, even more desirably from about 3 to about 6.5 atmospheres absolute, most desirably from about 4 to about 6.5 atmospheres absolute.

The at least perforable portion of the receptacle may be provided as a substantially planar portion, so as to minimize skidding or deflection of a firing pin (discussed below) as the firing pin contacts the portion. The at least perforable portion of the receptacle, which may advantageously be in the form of a foil, in particular a metal foil (e.g. an aluminum or a stainless steel foil), suitably has a thickness of at most 250 μm. For robustness of the receptacle, a thickness of at least 25 μm is favorable for the at least perforable portion, in particular a foil as described in the previous sentence. For enhanced robustness of the receptacle a thickness of at least 38 μm is desirable, more desirably of at least 50 μm. To further facilitate access to the receptacle e.g. through piercing, a thickness of at most 150 μm is desirable, more desirably of at most 100 μm, most desirably of at most 75 μm.

Returning to the exemplary embodiment shown in FIGS. 1 to 8, the receptacle may be held between a receptacle holder (2) and a carriage (3), whereby the perforable portion (1a) of the receptacle is positioned facing towards a firing pin (12) (see e.g. FIG. 7). The firing pin (12) desirably has an internal surface defining a channel along a single axis (X), wherein the channel has an opening at its first end positioned towards the receptacle and an opening at its second end positioned towards an outlet (9) adapted for insertion into a patient's mouth. Alternatively the outlet may be adapted for insertion into a patient's nasal cavity. Advantageously the channel may be generally conical from the first end to the second end. As can be seen in the exemplary embodiment, the outlet or mouthpiece (9), which is generally in the form of an elongated cylindrical component (as can be better seen in FIG. 4), has desirably a passageway extending along or parallel to the aforesaid axis (X). The outlet (9) may be affixed to the firing pin (12) for example through the use of a spacer (25) positioned therebetween (see also FIG. 4). The spacer may be an integral portion of the outlet (as in the exemplary embodiment and best seen in FIGS. 3 and 4), or an integral portion of the firing pin or a separate component affixed to the outlet and the firing pin or the spacer. Desirably the internal surface of the spacer (25), like that of the firing pin (12), defines a generally conical passageway from its first end towards the firing pin to its second end towards the outlet (9). The spacer (25) may include apertures or slits (15), the function of which is described in more detail below.

The outermost surface of the firing pin positioned towards the receptacle is suitably provided with a piercer, e.g. in the form of a sharp point. Referring to FIG. 14 showing schematically an enlargement of a portion of a tip (20) of a firing pin, the first end of the channel is advantageously slightly set back from the outermost surface of the firing pin and positioned adjacent to said piercer. FIG. 15 shows an enlargement of an alternative tip, in which an expansion chamber (50) and a spray break-up orifice (51) are provided.

The firing pin or the tip thereto is suitably made of a material, e.g. a material comprising a metal or a polymeric material, allowing perforation of the at least perforable portion of the receptacle. Surprisingly, it has been found that injection molded polymeric firing pins, such as those comprising polybutyleneterephthalate, acetal and/or polycarbonate, can be used to effectively perforate perforable portions of hermetically sealed receptacles, even when said portions are formed from metal (e.g. stainless steel or aluminum), e.g. metallic foil (such as 50 μm thick stainless steel foil). Alternative forms of piercer, such as oblique-cut stainless steel points are also suitable.

Returning to the exemplary embodiment as shown in FIGS. 1 to 8, the device may include a body or housing (8), within which is located a piston (5) that sits around a guide (4) mounted between a cap (7) and an outlet (9) (see FIG. 7). Air inlets (14), the function of which will be described below, may be provided between the cap (7) and the body (8) near the shoulder of the piston (5) facing towards the cap (see FIG. 2 in conjunction with FIGS. 7 and 8). Referring to FIG. 7, the firing pin (12) abuts the guide (4) and thus holds it in place. Suitably located within the guide (4) is the carriage (3) to which is clipped the receptacle holder (2) together with the receptacle (1). A compression spring (6) is desirably mounted between the cap (7) and the receptacle holder (2), such that the receptacle (1) and in particular the assembly of the receptacle holder (2), receptacle (1), and carriage (3) is biased towards the firing pin (12) (and thus away from the cap (7)).

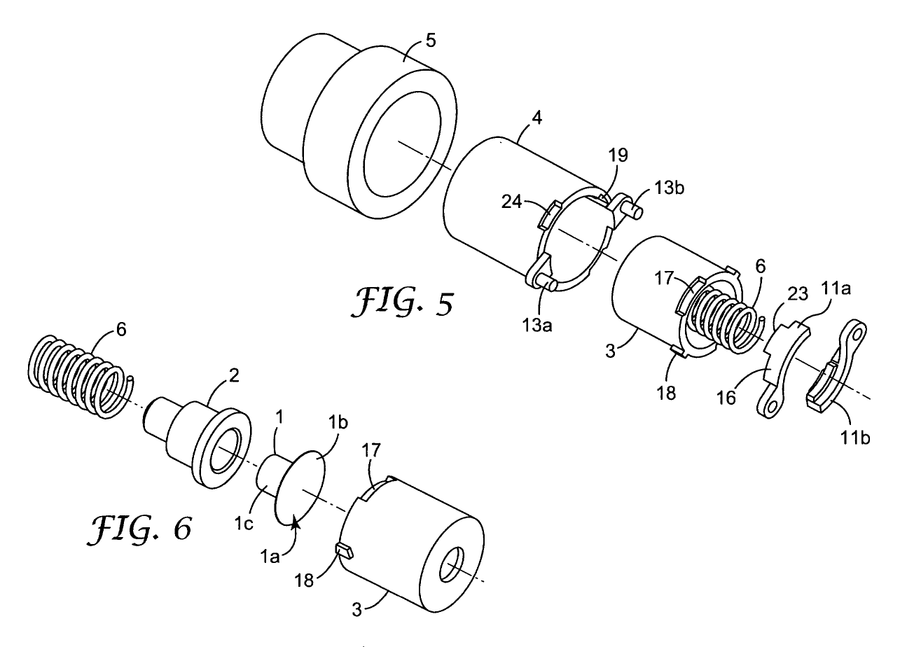

In the stored or as-supplied state of the device, the receptacle is suitably retained apart from the firing pin (i.e. cannot move towards the firing pin) through the use of a retention system. For example in the exemplary embodiment shown in FIGS. 1 to 8, as can be recognized from FIG. 7, the assembly (1, 2, and 3) cannot move as it is retained e.g. by a pair of catches (11a, 11b) that are radially pivotally mounted on two posts (13a, 13b, not visible in FIG. 7, but to be seen in FIGS. 3 and 5) that are part of the guide (4). The outer ends of these catches (11a, 11b) are desirably biased radially outwardly in a way that will be explained in more detail below. For actuation of the exemplary device, the receptacle (1), and in particular the receptacle-comprising assembly (1,2,3), is released upon a rotation of the catches (11a, 11b) on the posts (13a, 13b) as will be explained in more detailed below. However in its stored or as-supplied state the catches (11a, 11b) cannot rotate on the posts (13a, 13b) due to the presence of the piston (5).

Advantageously the device, as supplied to a patient, also comprises a fail-safe mechanism preventing any release of the retention system, e.g. a catch, until the patient is ready to use the device. In particular, the device may advantageously comprise a removable cover that suitably clips to the device, covering the outlet and includes a fail-safe mechanism preventing any release of the retention system, e.g. the catch. For example, in the exemplary embodiment shown in FIGS. 1 to 8, as can be seen in FIG. 7, the removable cover (10) clips to the body (8) covering the outlet (9) and projections (21) provided on the inner surface of the cover (10) pass through apertures (15) in the spacer (25) and abut the piston (5), holding the piston (5) immovably in place and thus prevent any rotation of the catches (11a, 11b). Advantageously the cover may also be configured and arranged to cover any air inlets (if present) of the device in order to prevent the ingress of fluff, dirt or other contaminants into the air inlets. For example, in the exemplary embodiment shown in FIGS. 1 to 8, the cover (10) also comprises long clips (22) that cover the air inlets (14) near the cap (7) of the device. See also e.g. FIGS. 1 and 2 showing perspective views of the exemplary embodiment in its stored or as-supplied state (FIG. 1) and its state upon removing the cover (10).

In the exemplary embodiment shown in FIGS. 1 to 8, the carriage (3) as well as the receptacle (1) and receptacle holder (2) are biased towards the firing pin (12) by the spring (6), as has already been explained. The carriage (3) is also biased rotationally (generally clockwise, as seen in FIG. 5) under the influence of the spring (6) when the inhaler is in its as-supplied state, through the provision of two angled teeth (18) on the outside of the carriage (3) that engage with the angled top profiles of two tracks (19) on the inside wall of the guide (4). The outer surface of the carriage (3) is also suitably provided with two recesses (17) that engage with protruding parts (16) of the catches (11a, 11b), and these recesses (17) have inclined surfaces at their ends, and thus apply a bias radially outwardly to the outer ends of the catches (11a, 11b). Further protruding parts (23) on the catches (11a, 11b) also engage with two recesses (24) on the guide (4).

To use the exemplary breath-actuated device shown in FIGS. 1 to 8, the patient first removes and discards the cover (10), thereby unlocking the piston (5). The piston does not move, however, due to frictional forces exerted radially outwardly on its inside from the two catches (11a, 11b). When the patient places the outlet, e.g. mouthpiece (9), in their mouth and starts to inhale through the device, a pressure difference is created between the regions in front of and behind the piston (5). When that pressure difference is sufficient, the piston is able to overcome the frictional resistance upon it due to the two catches (11a, 11b) and it moves forward, towards the patient. The movement of the piston (5) allows the two catches (11a, 11b) to pivot outwards on the posts (13a, 13b) under the influence of the aforementioned radially outward bias. Further movement of the piston (5) also uncovers the air inlets (14). As the protruding parts (16) on the catches (11a, 11b) disengage from recesses (17) on the carriage (3) (see FIGS. 5 and 8), the carriage becomes free to turn under the influence of the aforementioned rotational bias. Its two angled teeth (18) move down the angled profiles of the tops of the tracks (19) until the carriage (3) has rotated far enough for the teeth (18) to move axially down the tracks (19) under the influence of the spring (6). In other words, movement of the piston (5) due to inhalation allows release of the receptacle-comprising assembly (1,2,3), which is then rapidly accelerated towards the firing pin (12) under the influence of the spring (6). When the perforable portion (1a) of the receptacle (1) strikes the tip (20) of the firing pin (12), the foil ruptures, thus releasing the dose of pressurized formulation as an aerosol cloud through the channel of the firing pin (12). The dose passes down the passageway of the spacer (25), where it mixes with the inhaled air stream that flows from the inlets (14) and through the holes (15), through the passageway of and out of the outlet (9), so that the patient can thus inhale the dose.

The triggering inhalation pressure drop, at which the device fires to release the dose, may be appropriately selected by the manufacturer, for example through selection of the angles of the various interacting surfaces of the catches (11a, 11b), carriage (3), and guide (4).

As can be best appreciated from FIG. 7, desirably the inhalation device is arranged such that upon perforation of the said at least perforable portion of the receptacle by the firing pin the first end of the channel of the firing pin passes into a liquid portion of the pressurized formulation. In the exemplary embodiment shown in FIGS. 1 to 8 (as well as in the second exemplary embodiment discussed in detail below) the receptacle is centered (along axis X) relative to the firing pin. However the positioning of the receptacle relative to the firing pin may alternatively be off-centered, so that e.g. in the typical (generally horizontal) position of use of the device by the patient the firing pin perforates the receptacle such that first end of the firing pin channel passes into the liquefied portion of the pressurized formulation at or near the lower end thereof (away from the headspace).

The nature of the retention and release arrangements in the first exemplary embodiment, together with the in-line arrangement of the piston and carriage movements with the axis of the outlet, leads to a particularly small and compact device. Other retention and release structures are also possible.

A second exemplary embodiment in accordance with the present invention is shown in FIG. 9 to 13. This device is similar to the first exemplary embodiment shown in FIGS. 1 to 8 and differs mainly in that it is a “press-and-breathe” type inhaler, rather than a breath-actuated one.

Referring to FIGS. 12 and 13, this exemplary inhalation device is similar to the first exemplary embodiment in that it comprises a hermetically sealed receptacle (1) comprising at least a perforable portion (1a). That portion may advantageously be in the form of a foil (1b), in particular a metal foil, which is for example laser welded onto a receptacle body (1c), in particular a metal receptacle body. Again the receptacle contains a single dose of a pressurized formulation (30) comprising a pharmaceutically active ingredient and a liquefied aerosol propellant consisting of HFA 134a, HFA 227 or a mixture thereof. Again the receptacle may be held between a receptacle holder (2) and a carriage (3), whereby the perforable portion (1a) of the receptacle is suitably positioned facing towards a firing pin (12) (see e.g. FIGS. 12 and 13).

Also similar to the first embodiment the firing pin (12) advantageously has an internal surface defining a channel along a single axis (X), wherein the channel has an opening at its first end facing towards the receptacle and an opening at its second end facing towards an outlet (9). The outlet (9) is adapted for insertion into a patient's mouth. The channel of the firing pin is advantageously generally conical from the first end to the second end. Again, the outlet or mouthpiece (9), which is generally in the form of an elongated cylindrical component (as can be better seen in FIG. 9), has a passageway, which desirably extends along or parallel to the aforesaid axis (X). The outlet (9) may be affixed to the firing pin (12) for example through the use of a spacer (25) positioned therebetween. The internal surface of the spacer (25) desirably defines a generally conical passageway from its first end towards the firing pin to its second end towards the outlet (9). The spacer (25) may include apertures or slits (15).

Referring to FIGS. 12 and 13, this exemplary device also suitably includes a body or housing (8), within which is located a guide (4) mounted between a cap (7) and an outlet (9). Air inlets (14) may be provided between the cap (7) and the body (8) near the end of the guide facing towards the cap (see FIG. 10 in conjunction with FIGS. 12 and 13). As in the first exemplary embodiment, the firing pin (12), the tip (20) of which suitably comprises a piercer in the form of a sharp point with the opening at the first end of the channel desirably located adjacent thereto (see FIGS. 14 and 15), abuts the guide (4) and thus holds it in place, Suitably located within the guide (4) is the carriage (3) to which is clipped the receptacle holder (2) together with the receptacle (1). A compression spring (6) is desirably mounted between the cap (7) and the receptacle holder (2), such that the receptacle (1) and in particular the assembly of the receptacle holder (2), receptacle (1), and carriage (3) is biased towards the firing pin (12).

Referring to FIGS. 12 and 13, the receptacle (1), and in particular the receptacle containing assembly (1,2,3), is suitably retained apart from the firing pin (12) through the use of a catch (41) provided on the guide, whereby the catch (41) engages (and thus retains) a tab (42) provided on the carriage (3)

Similar to the first embodiment, this embodiment advantageously comprises a removable cover (10) that clips to the device, e.g. to the body (8), and covers the outlet (9). The cover desirably includes projections (21) that pass through holes (15) in the spacer (25) and abut the carriage (3), helping to hold it immovably in place. The cover (10) also has two long clips (22) that prevent the ingress of contaminants into air inlets (14) in the inhaler.

To use the device, the patient first removes and discards the cover (10), thereby uncovering the air inlets (14) (See FIG. 13). The patient then places the outlet (9) in their mouth and starts to inhale, thereby setting up an air-flow through the inlets (14) and through the holes (15) into the region within the spacer (25) and mouthpiece (9). At the same time, the patient presses on a button (40) that is integrally molded as part of the guide (4). Pressing on that button causes the catch (41) to pivot outwards, away from its engagement with the tab (42) on the carriage (3), allowing the carriage and thus the receptacle-containing assembly (1,2,3) to freely move under the influence of the spring (6). The receptacle-containing assembly (1,2,3) is thus rapidly accelerated towards the firing pin (12) to release the dose. The patient inhales the dose as a respirable aerosol mixed with the air stream through the inhalation device.

Whilst the two exemplary inhalation devices described above are intended to be fully disposable, i.e. non-refillable, it will be apparent to those skilled in the art that embodiments of the devices can readily be envisaged in which the device is provided e.g. with an access panel, so that the inside of the device can be accessed e.g. by the patient or a care-giver, in order to replace the receptacle or some sub-assembly of the device including the receptacle.

The above description provides but two examples of embodiments of inhalation devices. Alternative embodiments and features may be envisaged, such as the provision of features in the outlet region including baffles, as disclosed in EP 551 338 (to McAughey and Pritchard), or a large bowl arrangement, as disclosed in U.S. Pat. No. 5,115,803 (to Sioutas). FIG. 16 shows an outlet region in the form of a mouthpiece in which a baffle (55) is centrally held by supports (not shown). The pressurized formulation contained within the receptacle and comprising a pharmaceutically active ingredient and a liquefied aerosol propellant consisting of 1,1,1,2-tetrafluoroethane (HFA 134a), 1,1,1,2,3,3,3-heptafluoropropane (HFA 227) or a mixture thereof may optionally comprise one or more suitable pharmaceutical non-propellant and non-gaseous excipients, such as surfactants, preservatives, flavorings, antioxidants, anti-aggregating agents and co-solvents, e.g. ethanol. The term “excipients” as used herein means chemical agents having little or no pharmacological activity (for the quantities used) but which may enhance the pharmaceutical formulation or the performance of the inhalation device. Under the term “non-propellant excipient” is to be understood that the excipient is not a propellant. “Propellant” used herein means an inert liquid with a boiling point from about 25° C. to −43° C. which exerts a high vapor pressure at room temperature, e.g. a hydrocarbon (such as propane, butane, isobutane), a chlorofluorocarbon or a hydrogenated-chlorofluorocarbon. Under the term “non-gaseous” excipient is to be understood that the excipient is not a gas (i.e. a substance having a boiling point less than −43° C., e.g. carbon dioxide, oxygen, nitrogen).

It will be appreciated by those skilled in the art that the pharmaceutical pressurized formulation for use in the invention may contain a single pharmaceutically active ingredient or a combination two or more other pharmaceutically active ingredients.

Such pharmaceutically active ingredients may be selected from any suitable medicaments used in inhalation therapy. Appropriate medicaments may thus be selected from, for example,

analgesics, e.g. codeine, dihydromorphine, ergotamine, fentanyl or morphine; anginal preparations, e.g. diltiazem, nitroglycerin;

antiallergics, e.g. cromoglycate, ketotifen or nedocromil;

antiinfectives e.g. cephalosporins, penicillins, streptomycin, sulphonamides, tetracyclines and pentamidine;

antihistamines, e.g. methapyrilene;

anti-inflammatories, e.g. beclomethasone (e.g. the dipropionate), flunisolide, budesonide, ciclesonide, mometasone (e.g. the fuorate), fluticasone (e.g. the propionate) or triamcinolone acetonide;

antitussives, e.g. noscapine;

bronchodilators, e.g. salbutamol, salmeterol, ephedrine, adrenaline, fenoterol, formoterol, isoprenaline, metaproterenol, phenylephrine, phenylpropanolamine, pirbuterol, reproterol, rimiterol, terbutaline, isoetharine, tulobuterol, orciprenaline, or (−)-4-amino-3,5-dichloro-alpha-[[[6-[2-(2-pyridinyl)ethoxy]hexyl]amino]-methyl]benzenemethanol;

diuretics, e.g. amiloride;

antimuscarinics, e.g. anticholinergics such as ipratropium, atropine or oxitropium;

hormones, e.g. cortisone, hydrocortisone or prednisolone;

xanthines e.g. aminophylline, choline theophyllinate, lysine theophyllinate or theophylline;

phosphodiesterase inhibitors, e.g. PDE-4 inhibitors such as roflumilast; and

leukotrene modifiers, e.g. montelukast or zafirlukast. It will be clear to the person skilled in the art that where appropriate, the medicaments may be used as a free base or in a physiologically acceptable form, e.g. in the form of salts (e.g. as alkali metal or amine salts or as acid addition salts) or as esters (e.g. lower alkyl esters) or as solvates (e.g. hydrates).

Inhalation devices in accordance with the present invention are particularly advantageous for use in providing single doses of very expensive and/or very sensitive pharmaceutically active ingredients cost-effectively, e.g. especially for therapies including macromolecules (e.g. proteins and peptides) or other biological products. Accordingly, the pharmaceutically active ingredient may advantageously be selected from insulin, glucagon, g-csf (granulite colony simulating factor), erythropoietin, growth hormone, alpha-interferon, beta-interferon, calcitonin, alpha-1-anti-trypsin, oxytosin, somatostatin, parathyroid hormone, tnf (tumor-necrosis-factor)-alpha, Dnase, vasopressins (e.g. arginine vasopressin and ornithine vasopressin), LHRH analog, bovine-IgG, ferritin, gene transfer or therapy preparations (e.g. recombinant vectors (viral or non-viral), virus, naked or complex plasmids, virus producing cells, in vitro genetically modified cells, or portions of nucleic acids (e.g. for anti-sense type therapy)), somatic cell therapy preparations, molecules issued from rDNA and vaccines.

1. An inhalation device comprising a hermetically sealed receptacle containing a single dose of a pressurized formulation comprising a pharmaceutically active ingredient and a liquefied aerosol propellant consisting of HFA 134a, HFA 227 or a mixture thereof and wherein at least a portion of the receptacle is perforable.

2. An inhalation device according to claim 1, wherein the internal pressure within the receptacle is at most 7 atmospheres.

3. An inhalation device according to claim 1, wherein the internal volume of the receptacle is less than 0.3 ml.

4. An inhalation device as claimed in claim 3 wherein the internal volume of the receptacle is 0.2 ml or less.

5. (canceled)

6. (canceled)

7. An inhalation device according to claim 1, wherein the device further comprises a firing pin, wherein the firing pin comprises a channel, said channel having an opening at both ends, the first end positioned towards the receptacle, and wherein the receptacle and firing pin are mutually biased towards one another and wherein the device is arranged such that in the stored position the receptacle and firing pin are retained apart and upon actuation of the device said retention is released such that the receptacle and firing pin are mutually displaced to cause the firing pin to perforate the receptacle at said at least perforable portion and to cause aerosol formulation to pass through said channel and through to the patient.

8. An inhalation device according to claim 7, wherein the receptacle is biased towards the firing pin and the firing pin is held fixed within the device, and wherein the device is arranged, such that in the stored position the receptacle is retained apart from the firing pin and upon actuation of the device said retention is released such that the receptacle is displaced towards the firing pin to cause the firing pin to perforate the receptacle at said at least perforable portion and to cause aerosol formulation to pass through said channel and through to the patient.

9. An inhalation device according to claim 7, wherein the device is arranged such that upon perforation of said at least perforable portion of the receptacle by the firing pin the first end of the channel of the firing pin passes into a liquid portion of the pressurized formulation.

10. An inhalation device according to claim 7, wherein the firing pin has an internal surface and the internal surface defines the channel.

11. An inhalation device according to claim 10, wherein the internal surface of the firing pin defining the channel is arranged as to provide an expansion chamber.

12. An inhalation device according to claim 8, wherein the internal surface of the firing pin defining the channel is generally conical from the first end to the second end.

13. An inhalation device according to claim 7, wherein the outermost surface of the firing pin facing towards the receptacle is provided with a piercer capable of perforating said at least perforable portion of the receptacle and wherein the first end of the channel of the firing pin is set back from said outermost surface and positioned adjacent to said piercer.

14. An inhalation device according to claim 7, wherein the firing pin comprises a tip positioned towards the receptacle and the outer surface of the tip is conical in shape.

15. An inhalation device according to claim 7, wherein the device further comprises an outlet adapted for insertion into a patient's mouth or nose.

16. An inhalation device according to claim 15, wherein the channel of the firing pin extends substantially along a single axis and said second end of the channel is positioned towards the outlet, and wherein the outlet comprises a passageway, said passageway extending substantially along or being substantially parallel to said axis.

17. An inhalation device according to claim 16, wherein the outlet is affixed to the firing pin, optionally through the use of a spacer positioned therebetween.

18. An inhalation device according to claim 17, wherein the spacer has an internal surface and said internal surface defines a generally conical passageway from its first end towards the firing pin to its second end towards the outlet.

19. An inhalation device according to claim 1, wherein the device is actuated by breath actuation.

20. (canceled)

21. An inhalation device according to claim 1, wherein the receptacle is free of elastomeric seals and diaphragms and/or dispensing valves.

22. (canceled)

23. An inhalation device according to claim 1, wherein said at least perforable portion is substantially planar.

24. An inhalation device according to claim 1, wherein the at least perforable portion has a thickness of 250 μm at most.

25. (canceled)

26. (canceled)

27. (canceled)

28. (canceled)

29. (canceled)

30. (canceled)

31. An inhalation device according to claim 1, wherein the at least perforable portion is a foil.

32. An inhalation device according to claim 31, wherein the foil is laser welded to form a hermetic seal.

33. An inhalation device according to claim 1, wherein the at least perforable portion is metal.

34. An inhalation device according to claim 1, wherein the firing pin is made of a polymeric material.

35. (canceled)

35. (canceled)

37. (canceled)

38. (canceled)

39. An inhalation device according to claim 1, wherein the pharmaceutical active ingredient is a macromolecule or a biological therapy product.

40. (canceled)
